(19) United States
(12) Patent Application Publication
(10) Pub. No.: US 2003/0158521 A1
(43) Pub. Date: Aug. 21, 2003

(54) Trocar placement guide needle

(76) Inventor: Darius M. Ameri, Belmont, MA (US)

Correspondence Address: John A. Hamilton, III, Choate, Hall & Stewart, 53 State Street, Exchange Place, Boston, MA, 02109, US

(21) Appl. No.: 10/079,608

(22) Filed: Feb. 21, 2002

(51) Int. Cl. A61M 5/00

(52) U.S. Cl. 604/117; 606/167

(57) ABSTRACT

A trocar placement guide needle which reduces the need for trocar reinsertion attempts, by first ascertaining with the guide needle where a trocar should optimally be placed. In one embodiment, the guide needle is comprised of an elongate member including a proximal end and a sharp beveled distal tip for puncturing and cutting tissue, wherein the member includes an axial bore throughout the length of the member, a handle at the proximal end of the member, a means for indicating the depth to which the member has been inserted into the tissue, and a means for preventing unintended puncturing and cutting by the sharp beveled distal tip. Various embodiments are disclosed with different dimensions, means for precluding unintended puncturing or cutting, and depth indication means.

### FIELD OF THE INVENTION

The present invention relates generally to body cavity piercing devices used in surgery to provide access to internal cavities through small puncture sites rather than large incisions. More particularly, the present invention relates to a trocar placement guide for use in various endoscopic procedures, including abdominal or gynecological laparoscopy and thoracoscopy. A trocar placement guide needle according to the present invention is designed to be utilized with a variety of trocars so as to facilitate the initial introduction of the trocars into patients in a safe and controlled manner which provides greater angle and depth placement accuracy, minimizing the need for reinsertion attempts which in turn can cause trauma to the patient's abdominal wall, muscle and fascia, and unnecessary bleeding.

### BACKGROUND OF THE INVENTION

Abdominal laparoscopy, for example, is a form of surgery that involves the visualization of the interior of an abdominal cavity using an illuminating optical instrument, a laparoscope. The laparoscope and other instruments are introduced into the abdominal cavity through puncture orifices in the abdominal wall. An advantage of laparoscopic surgery includes the ability to conduct the procedures on an outpatient basis, thereby reducing patient and insurer medical costs. Another advantage is that surgeons are given the opportunity to view intra-abdominal viscera without performing a laparotomy, which requires a large incision of the abdominal wall. Small puncture wounds are created in laparoscopic surgery, lessening trauma. Additionally, laparoscopic procedures reduce postoperative patient discomfort, with recovery times measured in days as opposed to weeks. As these and other advantages are increasingly being recognized, the number and variety of laparoscopic procedures being performed are increasing.

In a typical abdominal laparoscopic procedure, a surgeon will usually introduce a Veress needle into the patient's abdominal cavity. The Veress needle has a stylet which permits the introduction of gas into the abdominal cavity. After the Veress needle is properly inserted, it is connected to a gas source and the abdominal cavity is insufflated to an approximate abdominal pressure of 15 mm Hg. By insufflating the abdominal cavity, pneumoperitoneum is created separating the wall of the body cavity from the internal organs. A trocar is then used to puncture the body cavity. A standard trocar is typically a pointed rod, usually constructed of metal and contained within a blunt-tipped sleeve known as a cannula. The trocar typically encloses an obturator having a beveled piercing tip having extremely sharp edges. The trocar is used to penetrate the abdominal wall with the assistance of the obturator, which is withdrawn from the cannula after the intra-abdominal end of the trocar is in the insufflated abdominal cavity. The cannula of the trocar remains in the body wall throughout the surgical procedure and instruments used during laparoscopic procedures, such as fiber optic cameras and the like, may be introduced into the abdomen through it. Trocars are available in different sizes to accommodate various surgical needs. Trocars can be utilized at different anatomical sites.

As technical knowledge is developed, improvements have been made in the design and construction of trocar cannulas, and surgical techniques involving the use of trocar cannulas in endoscopic procedures have likewise progressed. However, difficulties remain with respect to the proper placement of trocars. Trocar depth and angular positioning is crucial to the success of an operation. During an advanced endoscopic surgical procedure, the surgeon may, through the use of an endoscope, have a relatively good view of the targeted surgical site and multiple instruments the surgeon may be using with both hands. However, if the instruments were not inserted at proper angles, the surgeon may be forced to manipulate the instruments by contorting his wrists at uncomfortable angles. The insertion of these instruments are the result of the surgeon's estimations and are especially problematic in endoscopic surgery upon obese patients. Because the surgeon is unable to readily identify the specific internal organs which lie below a selected primary introduction site, the surgeon must also be exceedingly careful not to over insert the trocar and damage internal organs. Thus, a need exists to reduce reinsertion attempts which are presently required too often.

### SUMMARY OF THE INVENTION

The present invention provides a trocar placement guide needle which reduces the need for trocar reinsertion attempts, by first ascertaining with the guide needle where a trocar should optimally be placed. Benefits accruing as a result of the use of the trocar placement guide needle include, among others: less trauma, bleeding, and/or cosmetic damage to the patient; minimization of intraoperative conditions potentially leading to complication risks, such as extended anesthetic exposure or insufflation gas loss; lowered surgical cost, both in terms of intraoperative time and post-surgical recovery time; and more efficient, easier surgeries.

In one embodiment, the present invention provides a guide needle comprised of an elongate member including a proximal end and a sharp beveled distal tip for puncturing and cutting tissue, wherein the member includes an axial bore throughout the length of the member, a handle at the proximal end of the member, means for indicating the depth to which the member has been inserted into the tissue, and a means for preventing unintended puncturing and cutting by the sharp beveled distal tip.

In certain embodiments, a guide needle in accordance with the present invention has a substantially tubular shape. The guide needle is preferably in the range of 16 to 18 gauge and has an overall length of between 20 cm and 25 cm. As described below, in certain embodiments the guide needle provides a measure of what insertion depth a surgeon considered optimal.

In certain other embodiments, a guide needle in accordance with the present invention includes a blunt tipped stylet assembly for precluding unintended puncturing or cutting by the beveled distal tip of the elongate member. The blunt tipped stylet assembly may be either manually operable or naturally biased to a position which prevents the puncturing or cutting.

### BRIEF DESCRIPTION OF THE DRAWING

FIG. 1 is an illustration of one embodiment of a guide needle for facilitating trocar placement in accordance with the present invention.

FIGS. 2A, 2B are illustrations of cross-sectional and axial views depicting blunt stylet tip positions with respect to the end of a guide needle in accordance with one embodiment of the present invention.

FIG. 3 is an illustration of an axial view of a guide needle and pointer ring in accordance with one embodiment of the present invention.

### DETAILED DESCRIPTION OF CERTAIN EMBODIMENTS OF THE INVENTION

Certain embodiments of the invention will now be described with reference to the accompanying drawing.

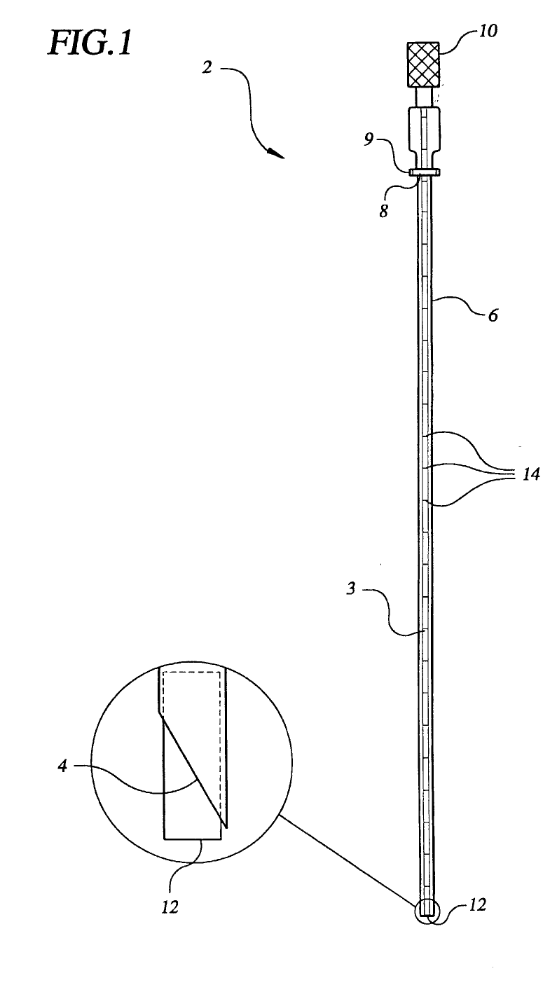

Referring to FIG. 1, the present invention provides a trocar placement guide needle 2 for greater accuracy in trocar insertions and placement. A trocar placement guide needle 2 in accordance with the present invention comprises: a elongated rod 3 composed of a rigid material, such as a metal, and having a hollow bore and a sharp cutting needle tip 4; a handle 10 or means for gripping the guide needle at the proximal end, such as a flange on the guide needle itself; and a means for preventing the sharp needle tip 4 from causing further damage after the desired tissue penetration has been accomplished.

During an abdominal laparoscopic procedure employing a guide needle 2 in accordance with the present invention, a surgeon positions the distal tip of the guide needle at a position where it is estimated a trocar is desired to be placed, and then pushes the guide needle into the patient until the insufflated abdominal cavity is pierced. The needle tip 4 includes a beveled edge that is sufficiently sharp to initially punctured and cut the tissue with minimal damage.

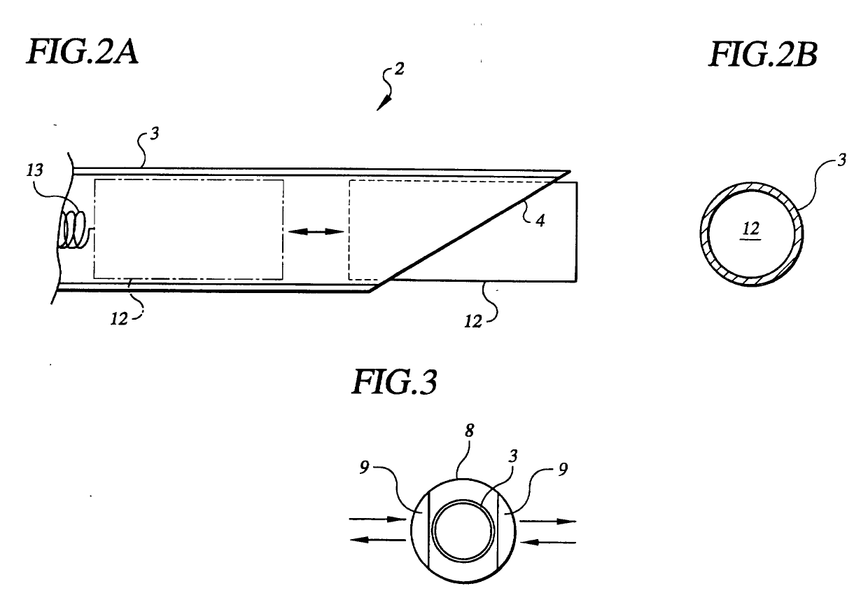

One means for preventing the needle tip 4 from causing damage after insertion is through the use of a stylet 12 (as depicted in FIGS. 2A and 2B) which is part of a stylet assembly and which is telescopically disposed through the hollow bore of the rod 3 and which has a blunt tip on its distal end. Note that the rod 3 is depicted as being tubular, however the rod's cross-sectional geometry is not intended to be limited to substantially circular shapes. A surgeon will generally receive an indication that the abdominal cavity has been penetrated, visually if the surgeon is employing viewing optics, or by a tactile sense of penetration resistance and/or an audible popping sound. After the cavity has been penetrated, the stylet 12 may be slideably extended to a first position beyond the needle tip 4 from its a second position within the hollow bore. The blunt tip of the stylet 12 will prevent the needle tip 4 from contacting tissue not intended to be cut. The stylet 12 also serves to reduce the amount of insufflating gas that may escape through the bore of the guide needle. In some embodiments, the stylet 12 may be biased toward the first position through some mechanism, perhaps by means of a spring 13 which propels the stylet into the first position when no tissue remains in front of the stylet providing resistance to stylet motion. Control of the extension of the stylet 12 to the first position may lie with a stylet control mechanism located at the handle 10. One skilled in the art will readily appreciate that there are several types of control mechanisms which can effect extension and retraction of the stylet 12.

The guide needle 2 may be comprised of any biocompatible material having sufficient structural strength to withstand the force required to push the guide needle through tissue. Suitable materials include certain plastics and surgical grade metals. Methods of manufacture typical for these materials (e.g., extrusion, molding) made be employed in forming the guide needle 2. It is preferred that the guide needle have a sufficiently small diameter, in the range of 16 to 18 gauge, in order to cause minimum trauma if the guide needle is not inserted in an optimal position and extraction and reinsertion is required. The guide needle will preferably have a length of 20 to 25 cm, providing sufficient length to penetrate the targeted anatomical site and to allow the surgeon to easily manipulate the guide needle. Due to the small diameter of the guide needle, a piercing made by the guide needle in the abdominal wall will largely self-seal after the guide needle is retracted, minimizing bleeding and the already remote potential for insufflation gas leakage. The cutting needle tip 4 of the needle will preferably have roughly a 45 degree angle beveled cutting edge at approximately the last 1 cm of the distal tip of the guide needle.

As described above, in a typical abdominal laparoscopy procedure, viewing optics are inserted into the abdominal area following insufflation. A surgeon will use the viewing optics to determine if the guide needle 2 has been optimally inserted, i.e., whether the surgeon would desire a trocar positioned precisely at the depth and angle at which the guide needle has been inserted. Once a proper insertion depth and angle have been determined, perhaps after several minimally traumatic guide needle penetrations, the insertion of a larger diameter trocar may be performed. Several techniques may be employed at this point. One method would include removing the guide needle completely, and reinserting the trocar in its place. Another method would include inserting coaxially the trocar along the path of the guide needle, and then retracting the guide needle. Other approaches are also within the scope of this invention.

Certain preferred embodiments of the invention provide a depth indication mechanism. The depth indication mechanism allows the surgeon to identify at what depth the inserted guide needle 2 is considered properly positioned. In certain preferred embodiments, the depth indication mechanism further comprises a combination of gradations 14 along the length of the guide needle and a pointer 8. The gradations 14 provide a visual indication to the surgeon of how deeply the guide needle has penetrated the patient. A variety of techniques may be employed in applying the gradations 14 to the rod 3, such as laser marking or ink printing processes. As depicted in FIG. 3, the pointer may comprise a ring slideably disposed around the rod 3. Once the surgeon determines that the guide needle 2 has been inserted to a proper depth, the pointer 8 may be slid down the length of the rod 3 to a point at which a particular gradation indicates the proper insertion depth. At this point, the pointer 8 is locked in place. A variety of pointers and means for locking the pointers at the appropriate gradation may be employed and are considered within the scope of the present invention. One such locking means provides that the pointer 8 is normally in a locked position, and that in order to move the pointer, one must unlock the pointer 8 for traversal down the rod 3, perhaps by squeezing a pair of tabs 9 on the pointer. By locking the pointer, the proper depth measurement is preserved without requiring the surgeon to make a mental or physical note. With this knowledge, the surgeon can avoid internal organs and more accurately select and position a trocar.

The guide needle 2 may additionally be enclosed in packaging 6, such as transparent or semi-transparent plastic in a tubular shape in order to prevent the guide needle 2 from being damaged or surgical personnel from being injured prior to use.

Other embodiments of the invention will be apparent to those skilled in the art from a consideration of the specification or practice of the invention disclosed herein. For example, although the foregoing description focuses on abdominal laparoscopic surgery, said description is not intended to be limiting. A guide needle in accordance with the present invention will benefit gynecologic laparoscopy and thoracoscopy. It is intended that the specification and examples be considered as exemplary only, with the true scope and spirit of the invention being indicated by the following claims.

What is claimed is:

1. A guide needle for improving trocar placement comprising:
an elongate member including a proximal end and a sharp beveled distal tip for puncturing and cutting tissue, wherein the member includes an axial bore throughout the length of the member;
a handle at the proximal end of the member;
means for indicating the depth to which the member has been inserted into the tissue; and
means for preventing unintended puncturing and cutting by the sharp beveled distal tip.

2. The guide needle of claim 1, wherein the member is substantially tubular in shape.

3. The guide needle of claim 2, wherein the member is preferably between 16 gauge and 18 gauge in diameter.

4. The guide needle of claim 1, wherein the member is preferably between 33 cm and 37 cm in length.

5. The guide needle of claim 1, wherein the depth indicating means comprises:
visible gradations at measured intervals along the member.

6. The guide needle of claim 5, wherein the depth indicating means further comprises:
a pointer mechanism lockable to the member at any visible gradation.

7. The guide needle of claim 6, wherein the pointer mechanism comprises a slideably movable ring disposed circumferentially about the member and including means for locking the position of the ring at any visible gradation along the member.

8. The guide needle of claim 1, wherein the preventing means comprises:
a stylet assembly telescopically disposed within the axial bore of the member, wherein the stylet assembly includes a blunt tip extendable to a first position beyond the beveled distal tip of the member and retractable to a second position within the axial bore.

9. The guide needle of claim 8, wherein the preventing means further comprises:
a stylet control mechanism located on or near the handle of the member for manually extending to the first position or retracting to the second position the stylet blunt tip.

10. The guide needle of claim 8, wherein the preventing means further comprising:
a spring biasing mechanism for automatically to the first position the stylet blunt tip when no opposing resistive force is applied to the stylet blunt tip.

11. The guide needle of claim 1, further comprising a guard enveloping the member, handle and stylet assembly.

12. A method for minimizing trauma to a patient undergoing laparoscopic surgery, comprising the steps of:
insufflating an anatomical target region;
inserting a narrow width guide needle with a depth indicator into the target region;
determining whether the insertion of the guide needle has resulted in optimal angular and depth positioning;
if the insertion is not optimal, extracting and reinserting the guide needle until optimal positioning has been achieved, then noting the optimal depth and extracting the guide needle; and
inserting a trocar in the precise position of guide needle insertion that was determined to be optimal.
